(19) United States
(12) Patent Application Publication
(10) Pub. No.: US 2024/0124869 A1
(43) Pub. Date: Apr. 18, 2024

(54) Curing disease by transcription regulatory gene editing

(71) Applicant: Koninklijke Nederlandse Akademie van Wetenschappen, Amsterdam (NL)

(72) Inventor: Wouter Leonard DE LAAT, Doorn (NL)

(73) Assignee: Koninklijke Nederlandse Akademie van Wetenschappen, Amsterdam (NL)

(21) Appl. No.: 18/264,587

(22) PCT Filed: Feb. 11, 2022
(86) PCT No.: PCT/EP2022/053341

§ 371 Date: Aug. 8, 2023

(30) Foreign Application Priority Data
Feb. 11, 2021 (EP) 21156461.2

(51) Int. Cl. C12N 15/10; C07K 14/805; C12N 9/22; C12N 15/11

(52) U.S. Cl. CPC C12N 15/102; C07K 14/805; C12N 9/22; C12N 15/11; C12N 2310/20; C12N 2800/80

(57) ABSTRACT

The invention pertains to an ex vivo method for increasing expression of at least one of gamma globin 1 (HBG1) and 2 (HBG2). The method can be used for the treatment or prevention of a haemoglobinopathy. The method comprises a step of introducing a first and a second site-specific endonuclease into a cell, wherein the first and second site-specific endonuclease generate a first and a second double stranded break in a chromosome, thereby generating a first, an intervening second and a third chromosomal fragment, wherein i) the first fragment comprises an beta globin locus control region (LCR) a functional fragment thereof; and ii) the third fragment comprises a sequence encoding at least one of HBG1 and HBG2. The length of the second intervening fragment is at least about 5 kb, and wherein joining the first fragment to the third fragment results in operably linking the beta globin LCR to the sequence encoding at least one of HBG1 and HBG2, thereby increasing at least one of HBG1 and HBG2 protein expression. The invention further pertains for compositions for use in the method of the invention.

### FIELD OF THE INVENTION

The present invention relates to the field of gene expression. More specifically, the invention provides compositions and methods for the controlling of gene expression through the excision and joining of specific chromosomal fragments.

### BACKGROUND

Many diseases known in the art are caused by aberrant gene expression and/or expression of a mutant protein having altered activity. Non-limiting examples include haemoglobinopathies, such as sickle cell disease (SCD) and beta-thalassemia (that),

In Sickle cell disease (SCD) point mutations in the coding parts of the adult beta-globin genes cause mis-folding of haemoglobin, forcing erythroid cells to adopt sickle-like shapes that block the smaller blood vessels. In beta-thalassemia (that) mutations may create premature stopcodons in the adult beta globin genes or may reduce the expression levels of the adult beta globin genes. This leads to an imbalance between the alpha-globin and beta-globin proteins, anemia and hyper-activation of blood cell production, in an attempt by the body to compensate for anemia. Both SCD and that are very severe diseases and current disease control is mostly through prenatal diagnosis.

Recently gene editing strategies were introduced and clinical trials are ongoing to test whether they may cure the disease. The strategies rely on isolation of CD34+ hematopoietic progenitor and stem cells (HPCs), targeted (CRISPR-Cas) editing and patient infusion with autologous edited HPCs (Zeng et al, Nat Med, 2020; 26(4):535-54). For editing, two approaches are followed. For SCD, base editing is applied to reverse the disease-causing mutation in the coding part of adult beta globin. For both SCD and that, BCL11A expression levels are reduced through the editing of the BCL11A enhancer (see e.g. Frangoul et al, N Engl J Med, 2021; 384(3):252-260). BCL11A is a repressor of fetal globin gene expression. By reducing the cellular amounts of BCL11A, fetal globin gene expression is reactivated and adult globin gene expression is reduced.

In erythroid cells, a powerful distal enhancer called the locus control region (LCR), composed of multiple DNase I hypersensitive sites (HSs), is in physical proximity to the beta-globin genes in a developmentally dynamic manner (Carter D et al, Nat Genet. 2002; 32:623-626, Tolhuis B et al, Molecular Cell. 2002; 10:1453-1465). Indeed, humans have a fetal stage specific beta-like globin (gamma-globin, or fetal beta-globin) genes that contact the LCR and are transcribed from the beginning of fetal liver erythropoiesis until birth when blood formation gradually shifts to the bone marrow.

The beta-globin gene cluster comprises in a sequential manner the LCR, the embryonic epsilon (ε)-globin, the fetal (HBG2 (Gγ) and HBG1 (Aγ))-globin and the adult (δ and β)-globin genes. It has been demonstrated previously that forced looping of the beta-globin LCR to the fetal globin genes in adult erythroid cells re-activates fetal globin gene expression and simultaneously downregulates adult globin gene expression (by bringing the LCR to the fetal genes it is hampered in its ability to contact and activate the adult globin genes) (Deng et al, Cell, 2014; 158(4):849-860). However forced looping requires introduction and stable expression of artificial looping factors, which may only transiently activate expression of the fetal globin, require a frequent treatment regime and hamper its potential for efficient treatment. There is therefore still a need in the art for an efficient treatment of haemoglobinopathies, in particular for the treatment of sickle cell disease and beta-thalassemia.

### SUMMARY

The invention may be summarized in the following embodiments:
Embodiment 1. An ex vivo method for increasing expression of a protein of interest, wherein the method comprises a step of introducing a first and a second site-specific endonuclease into a cell,
   wherein the first and second site-specific endonuclease generate a first and a second double stranded break in a chromosome, thereby generating a first, an intervening second and a third chromosomal fragment, wherein
   i) the first fragment comprises an enhancer sequence or a functional fragment thereof; and
   ii) the third fragment comprises a sequence encoding the protein of interest;
wherein the length of the second intervening fragment is at least about 5 kb, and wherein joining the first fragment to the third fragment results in operably linking the enhancer sequence to the sequence encoding the protein of interest, thereby increasing expression of the protein of interest.
Embodiment 2. The method of embodiment 1, wherein the cell is an hematopoietic stem and progenitor cell (HSPC), preferably a haematopoietic progenitor cell (HPC).
Embodiment 3. The method according to embodiment 1 or 2, wherein the first and second site-specific nuclease is selected from the group consisting of a CRISPR-nuclease complex, a zinc finger nuclease, a TALEN and a meganuclease.
Embodiment 4. The method according to embodiment 3, wherein the first and/or the second site-specific nuclease is a CRISPR-nuclease complex comprising at least one of a Cas9 protein and a single guide (sg)RNA.
Embodiment 5. The method according to any one of the preceding embodiments, wherein the genomic distance between the enhancer sequence and the sequence encoding the protein of interest is decreased at least about 20%, 30%, 40%, 50%, 60%, 70%, 80%, 90%, or at least about 95% as compared to the genomic distance between the enhancer sequence and the sequence encoding the protein of interest before introducing the first and second site-specific endonuclease.
Embodiment 6. The method according to any one of the preceding embodiments, wherein the protein of interest is at least one of gamma globin 1 (HBG1) and 2 (HBG2) and wherein the enhancer sequence is the beta globin locus control region (LCR).
Embodiment 7. The method according to embodiment 6, wherein the first fragment comprises LCR DNAse I hypersensitivity sites HS5, HS4, HS3, HS2 and HS1 and wherein the first double-stranded break is located less than about 300 bp from HS1.
Embodiment 8. The method according to embodiment 6, wherein the first double stranded break is located in the LCR, preferably wherein the first double stranded break is located within HS1, HS2 or HS3, or in between:
   i) LCR DNAse I hypersensitivity sites HS1 and HS2;
   ii) LCR DNAse I hypersensitivity sites HS2 and HS3; or
   iii) LCR DNAse I hypersensitivity sites HS3 and HS4;
preferably wherein the first double stranded break is located in between HS1 and HS2.
Embodiment 9. The method according to any one of embodiments 6-8, wherein the second double stranded break is located:
   i) at a position in between −1 to −1000 bp from the HBG1 transcription start site, preferably at a position in between −50 to −200 bp from the HBG1 transcription start site; or
   ii) at a position in between −200 to −700 bp from the HBG2 transcription start site.
Embodiment 10. The method according to any one of the preceding embodiments, wherein the wherein the length of the intervening second fragment is at least about 6, 7, 8, 9, 10, 11, 12, 13, 14 or at least about 15 kb.
Embodiment 11. A first and a second site-specific endonuclease, or one or more vectors encoding the same, for use in the treatment or prevention of a disease, wherein the first and second site-specific endonuclease can generate a first and a second double stranded break in a chromosome, thereby generating a first, an intervening second and a third chromosomal fragment, wherein
   i) the first fragment comprises an enhancer sequence or a functional fragment thereof; and
   ii) the third fragment comprises a sequence encoding a protein of interest;
wherein the length of the second intervening fragment is at least about 5 kb, and wherein joining the first fragment to the third fragment results in operably linking the enhancer sequence to the sequence encoding the protein of interest, thereby increasing expression of the protein of interest.
Embodiment 12. A first and a second site-specific endonuclease as defined in embodiment 11 for use in the treatment or prevention of a haemoglobinopathy, and wherein the protein of interest is at least one of HBG1 and HBG2.
Embodiment 13. A first and a second site-specific endonuclease for use according to embodiment 12, wherein the haemoglobinopathy is sickle cell disease or beta-thalassemia.
Embodiment 14. A method of treating a haemoglobinopathy, comprising the steps of
   isolating one or more haematopoietic stem and progenitor cells (HSPCs) from a subject suffering from a haemoglobinopathy;
   modifying the one or more HSPCs by introducing a first and a second site-specific endonuclease into the isolated HSPCs, wherein the first and second site-specific endonuclease generate a first and a second double stranded break in a chromosome, thereby generating a first, an intervening second and a third chromosomal fragment, wherein
      i) the first fragment comprises an enhancer sequence or a functional fragment thereof; and
      ii) the third fragment comprises a sequence encoding a protein of interest;
   wherein the length of the second intervening fragment is at least about 5 kb, and wherein joining the first fragment to the third fragment results in operably linking the enhancer sequence to the sequence encoding the protein of interest, thereby increasing expression of the protein of interest, wherein preferably the HSPCs are haematopoietic progenitor cells (HPCs); and
      administering the modified HSPCs to the subject.
Embodiment 15. A method according to embodiment 14, wherein the protein of interest is at least one of HBG1 and HBG2.
Embodiment 16. A method according to embodiment 14 or 15, wherein the method results in an increased fetal B-globin levels in the peripheral blood of the subject.

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

“A,” “an,” and “the”: these singular form terms include plural referents unless the content clearly dictates otherwise. The indefinite article “a” or “an” thus usually means “at least one”. Thus, for example, reference to “a cell” includes a combination of two or more cells, and the like.

“About” and “approximately”: these terms, when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

“And/or”: The term “and/or” refers to a situation wherein one or more of the stated cases may occur, alone or in combination with at least one of the stated cases, up to with all of the stated cases.

“Comprising”: this term is construed as being inclusive and open ended, and not exclusive. Specifically, the term and variations thereof mean the specified features, steps or components are included. These terms are not to be interpreted to exclude the presence of other features, steps or components.

Exemplary“: this terms means “serving as an example, instance, or illustration,” and should not be construed as excluding other configurations disclosed herein.

The terms “construct”, “nucleic acid construct”, “vector”, and “expression vector” are used interchangeably herein and is herein defined as a man-made nucleic acid molecule resulting from the use of recombinant DNA technology. These constructs and vectors therefore do not consist of naturally occurring nucleic acid molecules although a vector may comprise (parts of) naturally occurring nucleic acid molecules. A vector can be used to deliver exogenous DNA into a host cell, often with the purpose of expression in the host cell of a DNA region comprised on the construct. The vector backbone of a construct may for example be a plasmid into which a (chimeric) gene is integrated or, if a suitable transcription regulatory sequence is already present (for example a (inducible) promoter), only a desired nucleotide sequence (e.g. a coding sequence, an antisense or an inverted repeat sequence) is integrated downstream of the transcription regulatory sequence. Vectors may comprise further genetic elements to facilitate their use in molecular cloning, such as e.g. selectable markers, multiple cloning sites and the like. The vector backbone may for example be a binary or superbinary vector (see e.g. U.S. Pat. No. 5,591,616, US 2002138879 and WO 95/06722), a co-integrate vector or a T-DNA vector, as known in the art.

Expression vectors according to the invention are particularly suitable for introducing gene expression in a cell, preferably expression of a site-specific nuclease, preferably in a hematopoietic progenitor cell (HPC). A preferred expression vector is a naked DNA, a DNA complex or a viral vector, wherein the DNA molecule can be a plasmid. A preferred naked DNA is a linear or circular nucleic acid molecule, e.g. a plasmid. A plasmid refers to a circular double stranded DNA loop into which additional DNA segments can be inserted, such as by standard molecular cloning techniques. A DNA complex can be a DNA molecule coupled to any carrier suitable for delivery of the DNA into the cell. A preferred carrier is selected from the group consisting of a lipoplex, a liposome, a polymersome, a polyplex, a viral vector, a dendrimer, an inorganic nanoparticle, a virosome and cell-penetrating peptides.

The term “gene” means a DNA fragment comprising a region (transcribed region), which is transcribed into an RNA molecule (e.g. a pre-mRNA or ncRNA) in a cell. The transcribed region can be operably linked to suitable regulatory regions (e.g. a promoter), which form part of the gene as defined herein. A gene can comprise several operably linked fragments, such as a promoter, a 5′ leader sequence, a coding region and a 3′ non-translated sequence (3′ end) comprising a polyadenylation site. The gene may be part of a larger DNA molecule, such as a chromosome. The gene may be located within the genome. Preferably, the transcribed region encodes for a protein of interest.

“Expression of a gene” or “expression of a protein of interest” refers to the process wherein a DNA region is transcribed into an RNA, and subsequently translated into a protein or peptide.

The term “operably linked” refers to a linkage of polynucleotide elements in a functional relationship. A nucleic acid is “operably linked” when it is placed into a functional relationship with another nucleotide sequence. For instance, a promoter, enhancer, or other transcription regulatory sequence, is operably linked to a coding sequence if it affects the transcription of the coding sequence.

An “enhancer” is a stretch of nucleotides that can be recognized and bound by one or more proteins, preferably one or more transcription factors, to increase or induce the expression of one or more operably linked genes. A preferred enhancer for use in the invention is an Locus Control Region (LCR), preferably the beta-globin LCR.

“Promoter” refers to a nucleic acid fragment that functions to control the transcription of one or more nucleic acids. A promoter fragment is located upstream (5′) with respect to the direction of transcription of the transcription initiation site of the gene, and is structurally identified by the presence of a binding site for DNA-dependent RNA polymerase and transcription initiation site(s)

Optionally the term “promoter” may also include the 5′ UTR region (5′ Untranslated Region) (e.g. the promoter may herein include one or more parts upstream of the translation initiation codon of transcribed region, as this region may have a role in regulating transcription and/or translation).

“Sequence” or “Nucleotide sequence”: This refers to the order of nucleotides of, or within a nucleic acid. In other words, any order of nucleotides in a nucleic acid may be referred to as a sequence or nucleotide sequence.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide.

The term “complementarity” is herein defined as the sequence identity of a sequence to a fully complementary strand (defined herein below, e.g. the second strand). For example, a sequence that is 100% complementary (or fully complementary) is herein understood as having 100% sequence identity with the complementary strand and e.g. a sequence that is 80% complementary is herein understood as having 80% sequence identity to the (fully) complementary strand.

“Identity” and “similarity” can be readily calculated by known methods. “Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA). When sequences have a substantially different overall lengths, local alignments, such as those using the Smith Waterman algorithm, are preferred.

Alternatively percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

A “target sequence” is to denote an order of nucleotides within a nucleic acid that is to be targeted, e.g. the sequence recognized by a site-specific endonuclease. For example, the target sequence is an order of nucleotides comprised by a first strand of a DNA duplex.

An “endonuclease” is an enzyme that hydrolyses at least one strand of a duplex DNA upon binding to its recognition site. An endonuclease is to be understood herein as a site-specific endonuclease and the terms “endonuclease” and “nuclease” are used interchangeable herein. A restriction endonuclease is to be understood herein as an endonuclease that hydrolyses both strands of the duplex at the same time to introduce a double strand break in the DNA. A “nicking” endonuclease is an endonuclease that hydrolyses only one strand of the duplex to produce DNA molecules that are “nicked” rather than cleaved.

As used herein, the term “haemoglobinopathy” refers to a condition involving the presence of an abnormal haemoglobin molecule in the blood, or the absence of a normally expressed haemoglobin molecule in the blood.

Examples of haemoglobinopathies include, but are not limited to, SCD and THAL. SCD and THAL and their symptoms are well-known in the art and are described in further detail below. Subjects can be diagnosed as having a haemoglobinopathy by a health care provider, medical caregiver, physician, nurse, family member, or acquaintance, who recognizes, appreciates, acknowledges, determines, concludes, opines, or decides that the subject has a haemoglobinopathy.

The term “SCD” is defined herein to include any symptomatic anemic condition which results from sickling of red blood cells. Manifestations of SCD include: anemia; pain; and/or organ dysfunction, such as renal failure, retinopathy, acute-chest syndrome, ischemia, priapism, and stroke. As used herein the term “SCD” refers to a variety of clinical problems attendant upon SCD, especially in those subjects who are homozygotes for the sickle cell substitution in HbS. Among the constitutional manifestations referred to herein by use of the term of SCD are delay of growth and development, an increased tendency to develop serious infections, particularly due to pneumococcus, marked impairment of splenic function, preventing effective clearance of circulating bacteria, with recurrent infarcts and eventual destruction of splenic tissue. Also included in the term “SCD” are acute episodes of musculoskeletal pain, which affect primarily the lumbar spine, abdomen, and femoral shaft, and which are similar in mechanism and in severity. In adults, such attacks commonly manifest as mild or moderate bouts of short duration every few weeks or months interspersed with agonizing attacks lasting 5 to 7 days that strike on average about once a year. Among events known to trigger such crises are acidosis, hypoxia, and dehydration, all of which potentiate intracellular polymerization of HbS (J. H. Jandl, Blood: Textbook of Hematology, 2nd Ed., Little, Brown and Company, Boston, 1996, pages 544-545).

As used herein, “THAL” refers to a (hereditary) disorder characterized by defective production of hemoglobin. In one embodiment, the term encompasses hereditary anemias that occur due to mutations affecting the synthesis of haemoglobins. In other embodiments, the term includes any symptomatic anemia resulting from thalassemic conditions such as severe or beta.-thalassemia, thalassemia major, thalassemia intermedia, alpha.-thalassemias such as hemoglobin H disease. Beta.-thalassemias are caused by a mutation in the beta-globin chain, and can occur in a major or minor form. In the major form of beta.-thalassemia, children are normal at birth, but develop anemia during the first year of life. The mild form of beta.-thalassemia produces small red blood cells. Alpha-thalassemias are caused by deletion of a gene or genes HBA1 or HBA2, from the alpha globin chain.

By the phrase “risk of developing disease” is meant the relative probability that a subject will develop a haemoglobinopathy in the future as compared to a control subject or population (e.g., a healthy subject or population). For example, an individual carrying the genetic mutation associated with SCD, an A to T mutation of the adult beta-globin gene, and whether the individual in heterozygous or homozygous for that mutation increases that individual's risk.

### DETAILED DESCRIPTION

The invention relates to genome editing methods altering the chromosomal distance between genes and an enhancer in order to change the expression levels of these genes. The method of the invention can have several therapeutic applications. For example, haemoglobinopathies, including sickle cell disease (SCD) and beta-thalassemia (that), collectively the most common single gene disorders worldwide, can be treated by induced expression of the developmentally silenced fetal beta-globin genes.

The invention forces activation of the fetal beta-globin genes in adult erythroid cells through genetic deletion of sequences in between the fetal globin genes and their natural enhancer, the upstream beta-globin locus control region (LCR). Close linear juxtaposition of this strong enhancer and the fetal beta-globin genes will cause their expression to therapeutic levels. Through gene competition, it can simultaneously down-regulate the expression of the adult beta-globin genes.

Therefore in a first aspect, the invention pertains to a method for increasing expression of a protein of interest, wherein the method comprises a step of introducing a first and a second site-specific endonuclease into a cell, wherein the first and second site-specific endonuclease generate a first and a second double stranded break in a chromosome, thereby generating a first, an intervening second and a third chromosomal fragment, wherein
i) the first fragment comprises an enhancer sequence or a functional fragment thereof; and
ii) the third fragment comprises a sequence encoding the protein of interest;
wherein the length of the second intervening fragment is at least about 5 kb, and wherein joining the first fragment to the third fragment results in operably linking the enhancer sequence, or the functional fragment thereof, to the sequence encoding the protein of interest, thereby increasing protein expression.

The joining of the first and third, preferably “genomic” or “chromosomal”, fragments may be accomplished using the cell's own cellular repair machinery, such as by non-homologous end joining (NHEJ). Joining, or ligating, the fragment comprising the enhancer to the fragment comprising the sequence encoding the protein of interest results in the excision of the second, intervening, fragment that was located in between the fragment comprising the enhancer and the fragment comprising the sequence of interest. Consequently, the enhancer sequence and the sequence encoding the protein of interest are brought in closer proximity, resulting in increased expression of the protein of interest. Hence, the method as detailed herein is also a method for reducing the genomic distance between an enhancer sequence and a coding sequence.

It is understood herein that the first and second double-stranded break are located in the same DNA molecule, preferably in the same chromosome. Preferably, the first and second double-stranded break are located in chromosome 11.

The method may be an in vivo method. Preferably, the method is an ex vivo method, preferably an in vitro method. An “ex vivo” method is understood herein as a method that is carried out outside a living organism. A preferred ex vivo method is carried out outside a vertebrate, mammalian and/or primate body, preferably outside a human body. An “in vitro” method is understood herein as a method that is carried out outside a living organism, and in a controlled environment. A preferred in vitro method is carried out outside a vertebrate, mammalian and/or primate body, preferably outside a human body, and in a controlled environment.

Increased expression of a protein of interest as used herein includes, but is not limited to, induced expression of a protein of interest.

### Cell

The cell for use in a method of the invention may be any type of cell, such as, but not limited to a vertebrate, mammalian and/or primate cell. Preferably the cell is a human cell. The cell may be a differentiated cell, however preferably the cell is at least one of a stem cell and a progenitor cell. Preferably the cell of use in the method of the invention is a hematopoietic stem cell (HSC), preferably a hematopoietic progenitor cell (HPC). A preferred HPC is of the erythroid lineage, preferably a HPC expressing a CD34+ cell surface marker.

The cell for use in the method of the invention is preferably a HPC of the erythroid lineage, indicating that the cell may undergo erythropoiesis such that upon final differentiation it forms an erythrocyte or red blood cell (RBC). Such cells may originate from bone marrow hematopoietic progenitor cells. Upon exposure to specific growth factors and other components of the hematopoietic microenvironment, hematopoietic progenitor cells can mature through a series of intermediate differentiation cellular types, all intermediates of the erythroid lineage, into RBCs. Thus, hematopoietic stem cells of the “erythroid lineage,” give rise to at least one of erythrocytes, monocytes, macrophages, neutrophils, basophils, eosinophils, and megakaryocytes to platelets. Preferably, the hematopoietic stem cell for use in the method of the invention gives rise to at least erythrocytes.

Optionally, the hematopoietic progenitor cell for use in the method of the invention is collected from the group consisting of peripheral blood, cord blood, chorionic amniotic fluid, placental blood and bone marrow.

Optionally after the method of the invention, the hematopoietic progenitor cell is cryopreserved prior to use, for example, ex vivo expansion and/or implantation into a subject.

Optionally after the method of the invention, the hematopoietic progenitor cell culture expanded ex vivo prior to use, for example, cryopreservation, and/or implantation/engraftment into a subject

Optionally after the method of the invention, the hematopoietic progenitor cell is differentiated in culture ex vivo prior to use, for example, cryopreservation, and/or implantation/engraftment into a subject.

### Enhancer Sequence and Protein of Interest

The method of the invention results in bringing an enhancer sequence closer to a sequence encoding a protein of interest, i.e. decreasing the (genomic) distance between an enhancer sequence and a protein encoding sequence. To this end, a first double stranded break is generated in or close to an enhancer sequence and a second double stranded break is generated close to sequence encoding a protein of interest, preferably in the promoter controlling the expression of the protein of interest. Joining the two outer fragments, and thus removing the intervening middle or “second” fragment, results in a closer juxtaposition of the enhancer sequence and the protein-encoding sequence, thereby increasing the expression of the protein of interest.

The first double stranded break is preferably generated in or in close proximity of an enhancer sequence. A preferred enhancer is a locus control region (LCR). A “locus control region” is a long-range cis-acting regulatory element that confers high level of expression of linked genes.

A preferred enhancer sequence is the beta-globin locus control region (LCR). The beta globin LCR is composed of multiple DNAs I hypersensitivity sites, annotated as HS5, HS4, HS3, HS2 and HS1, wherein HS1 is located closest to the beta globin genes.

Preferably after generating the first double stranded break, the generated first fragment comprises beta-globin LCR DNAse I hypersensitivity sites HS5, HS4, HS3, HS2 and HS1. Preferably, the first double stranded break is located in close proximity of the HS1 region. Preferably, the distance between the HS1 and the first double-stranded break is less than about 300, 250, 200, 150, 100, 50, 40, 30, 20, 10, 5, 3 or 1 bp. Preferably, the distance between the HS1 and the first double-stranded break is about 300, 250, 200, 150, 100, 50, 40, 30, 20, 10, 5, 3 or 1 bp.

It is known in the art that the individual HS sites of the beta-globin LCR can act as an enhancer sequence (see e.g. Fraser et al, Genes Dev, 1993 7(1):106-13; Bender et al, Blood, 2012; 119(16):3820-7). Therefore, the first double-stranded break may be located in the beta-globin LCR.

Preferably, the first double stranded break is located within the beta-globin LCR DNAse I hypersensitivity site HS1, HS2, HS3 or HS4. Preferably, the first double stranded break is located within the beta-globin LCR DNAse I hypersensitivity site HS1. Preferably, the first double stranded break is located within the beta-globin LCR DNAse I hypersensitivity site HS2. Preferably, the first double stranded break is located within the beta-globin LCR DNAse I hypersensitivity site HS3. Preferably, the first double stranded break is located within the beta-globin LCR DNAse I hypersensitivity site HS4.

Preferably, the first double stranded break is located in between beta-globin LCR DNAse I hypersensitivity sites HS1 and HS2such that the first fragment comprises HS2, HS3, HS4 and HS5. Preferably, the first double stranded break is located in between beta-globin LCR DNAse I hypersensitivity sites HS2 and HS3, such that the first fragment comprises HS3, HS4 and HS5. Preferably, the first double stranded break is located in between beta-globin LCR DNAse I hypersensitivity sites HS3 and HS4, such that the first fragment comprises HS4 and HS5.

The second double-stranded break is preferably located in close proximity of the protein-encoding sequence. The second double-stranded break is preferably located in the promoter controlling the expression of the protein of interest. A preferred protein of interest is fetal beta globin, preferably at least one of HBG2 (Ggamma) and HBG1 (Agamma). The second double stranded break preferably results in a (“third”) fragment comprising the sequence encoding HBG1. Optionally the second double stranded break results in a (“third”) fragment comprising the sequence encoding HBG1 and the sequence encoding HBG2.

Preferably, the second double-stranded break is located in the promoter controlling the expression of HBG1 and/or located in the promoter controlling the expression of HBG2.

Preferably, the second double-stranded break is located at least in the promoter controlling the expression of HBG1. Preferably, the location of the second double stranded break is in between about −1 to −1000 bp from the HBG1 transcription start site, preferably in between about −20 to −600 bp, −30 to −500 bp, −40 to −400 bp, −50 to −300 bp or in between about −70 to −200 bp from the HBG1 transcription start site. Preferably, the location of the second double-stranded break is in between about −50 to −200 bp from the HBG1 transcription start site.

The double-stranded break may be located in a sequence that is unique for the HBG2 promoter. Preferably, the location of the second double stranded break is in between about −200 to −700 bp from the HBG2 transcription start site.

As detailed herein, the method of the invention results in a decrease of the genomic distance between the enhancer sequence and the sequence encoding a protein of interest, due to the removal of the second fragment located in between the first fragment (comprising the enhancer sequence) and the third fragment (comprising the sequence encoding the protein of interest).

The genomic distance between the enhancer sequence and the promoter sequence is preferably decreased at least about 5%, 10%, 20%, 30%, 40%, 50%, 60%, 70%, 80%, 95%, or at least 100% as compared to the genomic distance between the enhancer sequence and the endogenous gene before introducing the first and second site-specific endonuclease. A 100% decrease in the genomic distance is understood herein in that the enhancer sequence is located directly next to the transcription start site of the sequence of interest.

In addition or alternatively, the location of the first and second double-stranded break can be determined by determining the length of the excised (second) fragment. Preferably, the length of the fragment that is removed from the chromosome is about 5, 6, 7, 8, 9, 10, 11, 12, 13, 14, 15, 16, 17 kb or more. Optionally, the excised (second) fragment comprises a part or a complete binding site for a repressor, i.e. a binding site for a transcription factor that reduces or inhibits the expression of one or more operably linked genes. Optionally, the excised (second) fragment comprises a part or a complete binding site for the repressor BCL11A. Optionally, the excised (second) fragment comprises a part or a complete binding site for the repressor BCL11A located between −100 to −120 bp from the HBG1 transcription start site.

### Site-Specific Endonuclease

The method of the invention comprises a step of exposing the DNA to at least two site-specific nucleases. The skilled person understands that any site-specific nuclease can be suitable for use in the method of the invention. Suitable endonucleases and vectors used for the site-specific genomic modification of HSPCs are e.g. disclosed in Ferrari et al, Gene therapy using haematopoietic stem and progenitor cells, Nat Rev Genet, 2020; doi: 10.1038, which is incorporated herein by reference.

The first site-specific (endo)nuclease is preferably selected from the group consisting of a CRISPR-nuclease complex, a zinc finger nuclease, a TALEN and a meganuclease. Similarly, the second site-specific (endo)nuclease may be selected from the group consisting of a CRISPR-nuclease complex, a zinc finger nuclease, a TALEN and a meganuclease. Preferably, the nucleases used in the method of the invention are the same type of nuclease, for example, both the first and second double-stranded break is generated by a CRISPR-nuclease complex, a zinc finger nuclease, a meganuclease or a TALEN. Preferably, at least one of the first and second site-specific endonuclease is a CRISPR-nuclease complex.

An endonuclease is to be understood herein as an endonuclease that hydrolyses both strands of the duplex at the same time to introduce a double strand break in the DNA. The location of the double-stranded break is determined by the site-specific nuclease, preferably in combination with a guide RNA. It is well-known in the art how to design a site-specific nuclease to ensure that the nuclease cleaves at a specific location in the duplex DNA. Hence, the skilled person knows how to design a site-specific nuclease to cleave the DNA at the predetermined first or second location.

The cleavage site (the first location and the second location) is determined by the sequence that is targeted by the nuclease, i.e. the target sequence. The target sequence is, in general, defined by the nucleotide sequence on one of the strands on the double-helical nucleic acid.

In a preferred embodiment, at least one of the nucleases is selected from the group consisting of a CRISPR nuclease-complex, a TALEN, a zinc finger nuclease and a meganuclease. In a preferred embodiment at least one nuclease is a CRISPR nuclease-complex.

TALENs (Transcription activator-like effector nucleases) are targetable nucleases and are used to induce single- and double-strand breaks into specific DNA sites. The fundamental building block that is used to engineer the DNA-binding region of TALENs is a highly conserved repeat domain derived from naturally occurring TALEs encoded by Xanthomonas spp. proteobacteria. DNA binding by a TALEN is mediated by arrays of highly conserved 33-35 amino acid repeats that are flanked by additional TALE-derived domains at the amino-terminal and carboxy-terminal ends of the repeats. These TALE repeats specifically bind to a single base of DNA, the identity of which is determined by two hypervariable residues typically found at positions 12 and 13 of the repeat, with the number of repeats in an array corresponded to the length of the desired target nucleic acid, the identity of the repeat selected to match the target nucleic acid sequence. In some embodiments, the target sequence in the nucleic acid is between 15 and 20 base pairs in order to maximize selectivity of the target site. Cleavage of the target nucleic acid typically occurs within 50 base pairs of TALEN binding. Computer programs for TALEN recognition site design have been described in the art. See, e.g., Cermak et al, Nucleic Acids Res. 2011 July; 39(12): e82. Once designed to match the desired target sequence, TALENs can be expressed recombinantly and introduced into the cell as exogenous proteins, or expressed from a plasmid within the cell or administered as mRNA.

The site-specific nuclease may be a zinc finger nuclease. Zinc finger endonucleases combine a non-specific cleavage domain, typically that of Fokl endonuclease, with zinc finger protein domains that are engineered to bind to specific DNA sequences. The modular structure of the zinc finger endonucleases makes them a versatile platform for creating site-specific double-strand breaks to the genome. As Fokl endonuclease cleaves as a dimer, one strategy to prevent off-target cleavage events has been to design zinc finger domains that bind at adjacent 9 base pair sites. See also U.S. Pat. Nos. 7,285,416; 7,521,241; 7,361,635; 7,273,923; 7,262,054; 7,220,719; 7,070,934: 7,013,219: 6,979,539; 6,933,113; 6,824,978; each of which is herein incorporated by reference in its entirety.

The nuclease may be a meganuclease. The homing endonucleases, also known as meganucleases, are sequence specific endonucleases that generate double strand breaks in genomic DNA with a high degree of specificity due to their large (e.g., >14 bp) target sequence. Engineered homing endonucleases are generated by modifying the specificity of existing homing endonucleases. In one approach, variations are introduced in the amino acid sequence of naturally occurring homing endonucleases and then the resultant engineered homing endonucleases are screened to select functional proteins which cleave a targeted binding site. In another approach, chimeric homing endonucleases are engineered by combining the recognition sites of two different homing endonucleases to create a new recognition site composed of a half-site of each homing endonuclease. See e.g., U.S. Pat. Nos. 8,338,157.

Preferably, the nuclease is a CRISPR-Cas-derived editing agent. Such agents are known in the art and the skilled person readily understands that the method of the invention is not limited any specific editing agent. A preferred CRISPR-Cas-derived editing agent includes, but is not limited to, a Cas nuclease, a Cas transposase/recombinase and a prime editor. Suitable CRISPR-Cas-derived editing agents are e.g. disclosed in Anzalone et al (Nat Biotechnol. 2020; 38(7):824-844), which is incorporated herein by reference.

Preferably, the site-specific nuclease is a CRISPR nuclease complex, such as a CRISPR-Cas complex. The term CRISPR-nuclease, Cas, Cas-protein or Cas-like protein refers to CRISPR related proteins and includes but is not limited to CAS9, CSY4, Cas12, Cas13, Cascade, nickases (e.g. Cas9_D10A, Cas9_H820A or Cas9_H839A), Mad7 and fusion proteins (e.g. Cas9 or Cas-like molecules fused to a further functional domain such as an endonuclease domain) such as e.g. disclosed in Pickar-Oliver A. and Gersbach C A (Nat Rev Mol Cell Biol, 2019; 20(8):490-507), which is incorporated herein by reference, and other examples, such as Cpf1 or Cpf1_R1226A and such as for example described in WO2015/006747, WO2018/115390 and U.S. Pat. No. 9,982,279, which are incorporated herein by reference. Mutants and derivatives of Cas9 as well as other Cas proteins can be used in the methods disclosed herein. Preferably, such other Cas proteins have endonuclease activity and are able to recognize a target nucleic acid sequence when in a cell in the presence of a guide RNA that is engineered for recognition of the target sequence. The CAS-protein or CAS-like protein is preferable the CAS9 protein.

CAS or CAS-like protein may be, but is no limited to, selected from the group consisting of: Cas9 from Streptococcus pyogenes (e.g. UniProtKB—Q99ZW2), Cas9 from Francisella tularensis (e.g. UniProtKB—A0Q5Y3), Cas9 from Staphylococcus aureus (e.g. UniProtKB-J7RUA5), Cas9 from Actinomyces naeslundii (UniProtKB—J3F2B0), Cas9 from Streptococcus thermophilus (e.g. UniProtKB—G3ECR1; UniprotKB—Q03J16; Q03LF7), Cas9 from Neisseria meningitidis (e.g. UniProtKB—C9X1G5; UniProtKB—A1IQ68); Listeria innocua (e.g. UniProtKB—Q927P4); Cas9 from Streptococcus mutans (e.g. UniProtKB—Q8DTE3); Cas9 from Pasteurella multocida (e.g. UniProtKB—Q9CLT2); Cas9 form Corynebacterium diphtheriae (e.g. UniProtKB—Q6NK13); Cas9 from Campylobacter jejuni (e.g. UniProtKB—Q0P897), Cpf1 from Francisella tularensis (e.g. UniProtKB—A0Q7Q2), Cpf1 from Acidaminococcus sp. (e.g. UniProtKB—U2UMQ6), any orthologue thereof or any CRISPR associated endonuclease derived therefrom.

Preferred CRISPR-nuclease for use in the method of the invention is CRISPR-Cas9. In other embodiments, the Cas protein may be a homolog of Cas9 in which at least one of the RuvC, HNH, REC and BH domains is highly conserved.

A CRISPR-nuclease complex contains three basic design components: 1) a CRISPR-nuclease, such as Cas9; 2) a crRNA; and 3) a trans-activating crRNA (tracrRNA). In a preferred embodiment, the tracrRNA and crRNA may be combined in a single chain chimeric RNA (single guide RNA/sgRNA/gRNA).

The Cas9 protein is widely commercial available, as well as modified versions thereof (and which are also contemplated as CAS protein within the context of the current invention). The Cas9 protein has (endo)nuclease activity and is able to produce a specific DNA double strand break (DSB) at the target sequence.

The CRISPR-nuclease for use in the method of the invention may be Cpf1. Cpf1 is a single RNA-Guided Endonuclease of a Class 2 CRISPR-Cas System (Cell (2015) 163(3):759-771). Cpf1 is a single crRNA-guided endonuclease and it utilizes a T-rich protospacer-adjacent motif. Unlike Cas9, which requires crRNA and tracrRNA to mediate interference, Cpf1-crRNA complexes alone may cleave target DNA molecules, i.e. without the requirement for any additional RNA species. Cpf1 may thus be used as an alternative CAS-protein.

The CRISPR system comprises basically at least two entities: a “guide” RNA (gRNA) and a nonspecific CRISPR-associated endonuclease (e.g. Cas9 or Cpf1). The gRNA is a short RNA composed of a scaffold sequence necessary for Cas-binding and a user-defined nucleotide “targeting” sequence which defines the genomic target to be modified. Thus, one can change the genomic target of the CRISPR-nuclease (e.g. Cas9 or Cpf1) by simply changing the targeting sequence present in the gRNA. A guide RNA (gRNA) may be a crRNA hybridized to a tracrRNA, or a single chain guide RNA as described e.g. Jinek et al. (2012, Science 337: 816-820) when used in combination with e.g. the Cas9 nuclease. The gRNA is further to be understood to be a single RNA-guide (crRNA) such as for use with Cpf-1. Hence, the gRNA is the RNA molecule that directs the nuclease to a specific target sequence in the duplex DNA.

The invention as detailed herein specifies the use of a first and a second endonuclease to generate a first and second double-stranded break. The skilled person however readily understands that instead of an endonuclease, a combination of two nickases may be used to generate a double-stranded break, wherein the two nickases cleave opposite strands. A preferred nickase is a variant of a CRISPR-nuclease wherein one of the nuclease domains is mutated such that it is no longer functional (i.e., the nuclease activity is absent). A non-limiting example is a Cas9 variant having either the D10A or H840A mutation. A non-limiting example of a Cpf1 nickase is Cpf1 R1226A.

The guide RNA, when used in combination with e.g. Cas9, may be a fusion between a crRNA and a tracrRNA. It is however also contemplated within the invention that instead of a single sgRNA, a tracrRNA and a crRNA as separate RNA molecules can be used in combination with e.g. Cas9.

As indicated above, the CRISPR system requires at least two basic components, a CRISPR nuclease and a guide RNA. The skilled person knows how to prepare the different components of the CRISPR-nuclease system. In the prior art numerous reports are available on its design and use. See for example the review by Haeussler et al (J Genet Genomics. (2016)43(5):239-50) on the design of sgRNA and its combined use with the CAS protein CAS9 (originally obtained from S. pyogenes).

Preferably, the first and/or the second site-specific nuclease is a CRISPR-nuclease complex comprising at least one of a Cas9 protein and a single guide (sg)RNA.

### Treatment

In a further aspect, the invention pertains to a first and a second site-specific endonuclease, or one or more vectors encoding the same, for use in the treatment or prevention of a disease, wherein the first and second site-specific endonuclease can generate a first and a second double stranded break in a chromosome, thereby generating a first, an intervening second and a third chromosomal fragment, wherein
i) the first fragment comprises an enhancer sequence or a functional fragment thereof; and
ii) the third fragment comprises a sequence encoding a protein of interest;
wherein the length of the second intervening fragment is at least about 5 kb, and wherein joining the first fragment to the third fragment results in operably linking the enhancer sequence to the sequence encoding the protein of interest, thereby increasing expression of the protein of interest.
   Preferably, the protein of interest is at least one of HBG1 and HBG2. The one or more vectors encoding the first and second site specific nuclease are preferably gene therapeutic vectors. A preferred gene therapeutic vector is capable of infecting or transfecting a hematopoietic progenitor cell (HPC). A preferred vector is a viral vector, such as, but not limited to a lentiviral vector, an adeno-associated virus (AAV) vector or a retroviral vector, preferably a gammaretroviral vector. Such gene therapeutic vector are well-known in the art for gene therapy using haematopoietic stem and progenitor cells (Ferrari et al, Gene therapy using haematopoietic stem and progenitor cells, Nat Rev Genet, 2020; doi: 10.1038).

As used in the context of the invention, the terms “prevent”, “preventing”, and “prevention” refers to the prevention or reduction of the recurrence, onset, development or progression of a disease, preferably a haemoglobinopathy, preferably a haemoglobinopathy as defined herein, or the prevention or reduction of the severity and/or duration of the haemoglobinopathy or one or more symptoms thereof.

As used in the context of the invention, the terms “therapies” and “therapy” can refer to any protocol(s), method(s) and/or agent(s), preferably as specified herein below, that can be used in the prevention, treatment, management or amelioration of a disease, preferably a haemoglobinopathy, preferably a haemoglobinopathy as defined herein, or one or more symptoms thereof.

As used herein, the terms “treat”, “treating” and “treatment” refer to the reduction or amelioration of the progression, severity, and/or duration of a haemoglobinopathy, preferably a haemoglobinopathy as defined herein below, and/or reduces or ameliorates one or more symptoms of the disease.

The first and a second site-specific endonuclease, or one or more vectors encoding the same, may be comprised in a composition, preferably a pharmaceutical composition.

A used herein “compositions”, “products” or “combinations” useful in the methods of the present invention include those suitable for various routes of administration, including, but not limited to, intravenous, subcutaneous, intradermal, subdermal, intranodal, intratumoral, intramuscular, intraperitoneal, oral, nasal, topical (including buccal and sublingual), rectal, vaginal, aerosol and/or parenteral or mucosal application. The compositions, formulations, and products according to the disclosure invention normally comprise the drugs (alone or in combination) and one or more suitable pharmaceutically acceptable excipients.

Preferably, the first and second site-specific endonuclease as defined herein are for use in the treatment or prevention of a haemoglobinopathy.

Preferably, the one or more vectors encoding the first and second site-specific endonuclease as defined herein are for use in the treatment or prevention of a haemoglobinopathy. It is understood herein that when the first and second site-specific endonuclease is a CRISPR-nuclease complex, the same or additional vectors encode the endonuclease and the guide RNA as defined herein.

Preferably, the haemoglobinopathy is at least one of sickle cell disease and beta-thalassemia.

The medical use herein described is formulated as a combination of a first and a second site-specific endonuclease, or one or more vectors encoding the same, as defined herein for use as a medicament for treatment of the stated disease(s), but could equally be formulated as a method of treatment of the stated disease(s) using a combination as defined herein, a combination as defined herein for use in the preparation of a medicament to treat the stated disease(s), and use of a combination as defined herein for the treatment of the stated disease(s) by administering an effective amount, Such medical uses are all envisaged by the present invention.

As used herein, the term “effective amount” refers to the amount of the agent, i.e. of a combination of a first and a second site-specific endonuclease as defined herein, or one or more vectors encoding the same, which is sufficient to reduce the severity, and/or duration of a haemoglobinopathy, ameliorate one or more symptoms thereof, prevent the advancement of the haemoglobinopathy, or cause regression of the haemoglobinopathy, or which is sufficient to result in the prevention of the development, recurrence, onset, or progression of the haemoglobinopathy or one or more symptoms thereof.

The effective amount of active agent(s) used to practice the present invention for therapeutic treatment of a haemoglobinopathy varies depending upon the manner of administration, the age, body weight, and general health of the subject. Ultimately, the attending physician or veterinarian will decide the appropriate amount and dosage regimen. Such amount is referred to as an “effective” amount. Thus, in connection with the administration of the agent which, in the context of the current disclosure, is “effective against” a haemoglobinopathy indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

The invention further pertains to a method of treatment, comprising the steps of
isolating one or more haematopoietic stem and progenitor cells (HSPCs) from a subject;
modifying the one or more HSPCs by introducing a first and a second site-specific endonuclease into the isolated HSPCs, wherein the first and second site-specific endonuclease generate a first and a second double stranded break in a chromosome, thereby generating a first, an intervening second and a third chromosomal fragment, wherein
i) the first fragment comprises an enhancer sequence, or a functional fragment thereof; and
ii) the third fragment comprises a sequence encoding a protein of interest;
wherein the length of the second intervening fragment is at least about 5 kb, and wherein joining the first fragment to the third fragment results in operably linking the enhancer sequence to the sequence encoding the protein of interest, thereby increasing expression of the protein of interest; and
administering the modified HSPCs to the subject.

Preferably, the protein of interest is at least one of HBG1 and HBG2.

Preferably the HSPCs are haematopoietic progenitor cells (HPCs). Preferably, the method is for the treating a haemoglobinopathy and wherein the subject suffers from a haemoglobinopathy. The haemoglobinopathy is preferably at least one of sickle cell disease and beta-thalassemia.

The HSPCs may be collected from the group consisting of peripheral blood, cord blood, chorionic amniotic fluid, placental blood and bone marrow. Any method for isolating and modifying the HSPCs ex vivo is suitable for use in the method of the invention. Such method is for example disclosed in Zeng et al (Therapeutic base editing of human hematopoietic stem cells, Nat Med. 2020 Apr.; 26(4):535-541), which method is incorporated herein by reference.

The introduction of the first and second endonuclease can be done using any conventional method known in the art. The proteins may be delivered by transfecting the cells with the proteins. In case the first and second site-specific nuclease is a CRISPR-nuclease complex, they may be delivered by direct delivery of the CRISPR-nuclease complex. The site directed nuclease may be delivered by conventional means, such as, but not limited to PEG-mediated transfection and electroporation. In addition or alternatively, the site-specific nuclease can be expressed in the HSPC, e.g. by delivery of the sequence encoding the site-specific nuclease, optionally in combination with (a vector expressing) one or more guide RNAs.

The skilled person understands that the invention as detailed herein is not limited to two site-specific endonucleases. For example, additional endonucleases may be used to introduce additional double-stranded breaks in the second intervening fragment.

Administering or “returning” the HSPC to the subject preferably results in increased levels of fetal beta-globin in the peripheral blood of the subject. These increased levels preferably treat or prevent the haemoglobinopathy.

### FIGURE LEGENDS

FIG. 1. A) Schematic overview of the experimental set up, the positions of the LCR and GFP coding sequence is indicated. B) GFP expression levels and C) GFP expression levels over a time period of 3 weeks.

FIG. 2. A) Schematic overview showing binding sites for the CRISPR-Cas complex, to create cuts at the HBG genes (left) and in the LCR (right), upstream and inside HS2 of the LCR. The location of the HBG primer and HS2 primer is indicated with an arrow. B) Schematic representation of the guides used to create deletions. C) PCR using the HBG and HS2 primer (as shown in 2A)), demonstrating that deletions are made in K562 cells using guide combination 3-4, 3-5 and 3-7.

### EXAMPLES

### Example 1

To determine how genomic distance affects the functionality of the beta globin LCR to enhance gene expression, we used a GFP reporter gene driven by the HBG1/2 promoter, and a micro-LCR (with HS1234, see Talbot D, et al. Nature 1989, 338:352-5). The gene and uLCR are ectopically integrated in the genome of K562 cells, with the uLCR each time placed at a different distance from the GFP reporter gene.

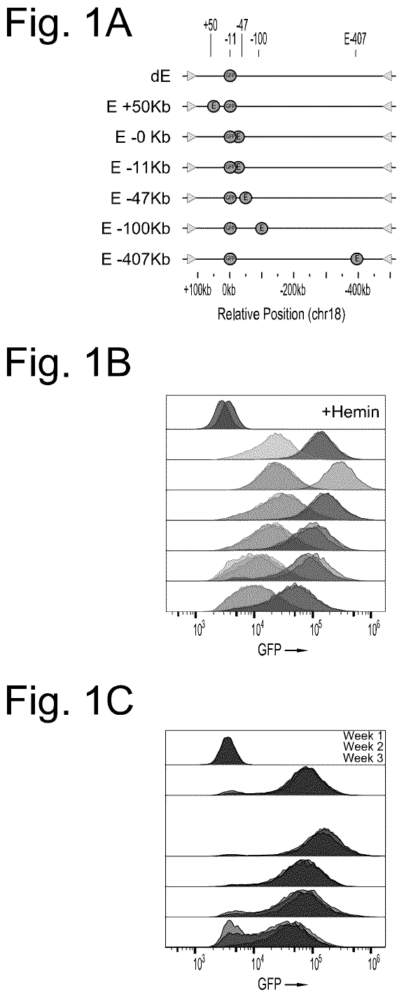

FIG. 1B shows the transcriptional output (GFP levels) of the reporter gene, before and after hemin-induced maturation of K562 cells (human erythroleukemia cells). This clearly shows that transcriptional output increases with decreased enhancer distance.

FIG. 1C shows a time course experiment over 3 weeks, demonstrating that cells start silencing the reporter gene when the uLCR is at large distance (407 kb, 100 kb), while a more proximal uLCR (11 kb, 47 kb) protects the reporter gene from silencing. Indeed, protection against silencing is most effective with the uLCR immediately next to the gene (0 kb) (data not shown). Further confirmation of this finding is also shown in Rinzema et al, bioRxiv, Oct. 5, 2021, DOI 10.1101/2021.10.05.463209 (incorporated herein by reference), teaching that the deletion of a ˜400 kb chromosomal segment in between a micro-LCR and a silenced HBG promoter-driven reporter gene indeed causes reactivation of the HBG promoter-driven reporter gene, leading to much higher expression levels of the reporter gene than seen with the ˜400 kb chromosomal segment in between the micro-LCR and the HBG promoter-driven reporter gene (Rinzema et al, in particular FIGS. 1(f) and 1(g)).

Hence in a series of experiments, we altered the distance between a fetal globin gene and the LCR, and we observed that (1) expression levels increase with decreased linear distance between the LCR and close linear juxtaposition of the endogenous LCR to the fetal globin genes, accomplished through genetic deletion of intervening sequences, reactivates the fetal globin genes, e.g. to levels that may cure SCD and that. This offers a universal strategy for curing SCD and Thal, independent of the exact disease-causing mutation in/around the adult globin genes.

### Example 2

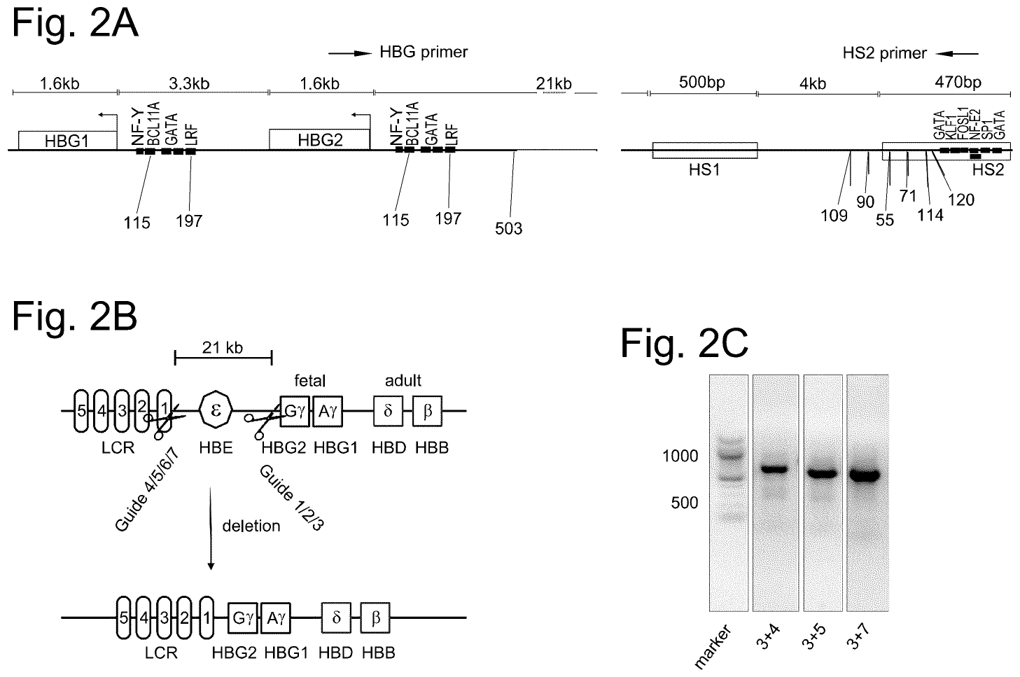

Several guide RNA combinations were evaluated for their ability to enable Cas9-mediated DNA simultaneous cutting near or inside an HBG promoter and near or inside HS2 of the LCR in K562 cells. The simultaneous cutting is expected to cause the deletion of the intervening chromosomal segment and thereby the immediate juxtaposition of the HS2-HS5 of the LCR to an HBG gene promoter. A schematic representation of the experiment is depicted in FIGS. 2A and 2B. The guide sequences are indicated in Table 1.

TABLE 1
Guide sequences for targeting
the HBG gene and the LCR (HS2)

| HBG/ | Guide | | | SEQ |
|---|---|---|---|---|
| HS2 | no. | | Guide sequence | ID NO |
| | | Position | | |
| | | from TSS | | |
| HBG | 1 | −115 | CTTGTCAAGGCTATTGGTCAAGG | 1 |
| | 2 | −197 | ATTGAGATAGTGTGGGGAAGGGG | 2 |
| | 3 | −503 | TAGTCTCATTTAGTAAGCAT | 3 |
| | | Position | | |
| | | from HS2 | | |
| HS2 | 4 | 109 | GTGGCCTCTATACCTAGAAG | 4 |
| | 5 | −55 | CTTTATGATGCCGTTTGAGG | 5 |
| | 6 | −71 | GAGGTGGAGTTTTAGTCAGG | 6 |
| | 7 | −114 | CAGGATGCTTACAGGGCAGA | 7 |
| | 8 | −120 | GCCCTGTAAGCATCCTGCTG | 8 |

Combinations of HBG guides and HS2 guides were introduced in K562 cells, together with CRISPR-Cas9, to create the desired deletions. PCR with one HBG primer and one HS2 primer confirmed the deletion as obtained with HBG guide no. 3 in combination with HS2 guides no. 4,5 or 7 (FIG. 2C). The skilled person readily understands that other combinations of guide sequences will be equally suitable for creating the desired deletions.

As a non-limiting example, the combination of guide RNA no. 3 and 7 was further evaluated in human primary pro-erythroblast cells. As indicated in Table 1, guide RNA no. 3 enables Cas9-mediated cutting near a HBG promoter and guide RNA no. 7 enables Cas9-mediated cutting inside HS2 of the LCR. Sanger sequencing confirmed that individual alleles indeed showed the presence of indels, demonstrating efficient CRISPR-Cas9 cutting at their respective target sites in primary pro-erythroblast cells.

1. An method for increasing expression of gamma globin 1 (HBG1) and/or 2 (HBG2), wherein the method comprises a step of introducing a first and a second site-specific endonuclease into a cell,
wherein the first and second site-specific endonuclease generate a first and a second double stranded break in chromosome 11, thereby generating a first, an intervening second and a third chromosomal fragment, wherein
i) the first fragment comprises an beta globin locus control region (LCR), or a functional fragment thereof; and
ii) the third fragment comprises a sequence encoding HBG1 and/or HBG2;
wherein the first double stranded break is located in or downstream of the LCR,
wherein the second double-stranded break is located upstream of the HBG1 transcription start site,
wherein the length of the intervening second fragment is at least 6 kb,
and wherein joining the first fragment to the third fragment results in decreasing the genomic distance and operably linking the beta globin LCR to the sequence encoding HBG1 and/or HBG2, thereby increasing HBG1 and/or HBG2 protein expression.

2. The method of claim 1, wherein the cell is an hematopoietic stem and progenitor cell (HSPC), preferably a haematopoietic progenitor cell (HPC).

3. The method according to claim 1, wherein the first and second site-specific endonuclease is selected from the group consisting of a CRISPR-nuclease complex, a zinc finger nuclease, a TALEN and a meganuclease.

4. The method according to claim 3, wherein the first and/or the second site-specific endonuclease is a CRISPR-nuclease complex comprises a Cas9 protein and/or a single guide (sg)RNA.

5. The method according to claim 1, wherein the genomic distance between the LCR and the sequence encoding HBG1 and/or HBG2 is decreased at least 30%, 40%, 50%, 60%, 70%, 80%, 90%, or at least 95% as compared to the genomic distance between the LCR and the sequence encoding HBG1 and/or HBG2 before introducing the first and second site-specific endonuclease.

6. The method according to claim 1, wherein the first fragment comprises LCR DNAse I hypersensitivity sites HS5, HS4, HS3, HS2 and HS1 and wherein the first double-stranded break is located less than 300 bp from HS1.

7. The method according to claim 1, wherein the first double stranded break is located in the LCR, preferably wherein the first double stranded break is located within HS1, HS2 or HS3, or in between:
i) LCR DNAse I hypersensitivity sites HS1 and HS2;
ii) LCR DNAse I hypersensitivity sites HS2 and HS3; or
iii) LCR DNAse I hypersensitivity sites HS3 and HS4;
preferably wherein the first double stranded break is located in between HS1 and HS2.

8. The method according to claim 1, wherein the second double stranded break is located
i) at a position in between −1 to −1000 bp from the HBG1 transcription start site, preferably at a position in between −50 to −200 bp from the HBG1 transcription start site; or
ii) at a position in between −200 to −700 bp from the HBG2 transcription start site.

9. The method according to claim 1, wherein the length of the intervening second fragment is at least about 7, 8, 9, or at least about 10 kb.

10. The method according to claim 4, wherein the first CRISPR-nuclease complex comprises a Cas9 protein and a guide RNA, wherein the guide RNA comprises a sequence selected from the group consisting of SEQ ID NO: 4, 5, 6, 7 and 8, and wherein the second CRISPR-nuclease complex comprises a Cas9 protein and a guide RNA, wherein the guide RNA comprises a sequence selected from the group consisting of SEQ ID NO: 1, 2 and 3.

11. The method according to claim 10, wherein the first CRISPR-nuclease complex comprises a guide RNA comprising a sequence having SEQ ID NO: 7 and the second CRISPR-nuclease complex comprises a Cas9 protein and a guide RNA, wherein the guide RNA comprises a sequence selected from the group consisting of SEQ ID NO: 1, 2 and 3.

12. The method according to claim 11, wherein the first CRISPR-nuclease complex comprises a guide RNA comprising a sequence having SEQ ID NO: 7 and the second CRISPR-nuclease complex comprises a guide RNA comprising a sequence having SEQ ID NO: 3.

13.-15. (canceled)

16. An ex vivo method for modifying one or more HSPCs, comprising the steps of
isolating one or more haematopoietic stem and progenitor cells (HSPCs) from a subject suffering from a haemoglobinopathy; and
modifying the one or more HSPCs by introducing a first and a second site-specific endonuclease into the isolated HSPCs, wherein the first and second site-specific endonuclease generate a first and a second double stranded break in a chromosome, thereby generating a first, an intervening second and a third chromosomal fragment, wherein
i) the first fragment comprises a beta globin locus control region (LCR) or a functional fragment thereof; and
ii) the third fragment comprises a sequence encoding HBG1 and/or HBG2;
wherein the length of the second intervening fragment is at least 6 kb, and wherein joining the first fragment to the third fragment results in operably linking the beta globin LCR to the sequence encoding HBG1 and/or HBG2, thereby increasing HBG1 and/or HBG2 protein expression.

17. The method according to claim 16, wherein the HSPCs are haematopoietic progenitor cells (HPCs).

18. A method for the treatment of prevention of a haemoglobinopathy, the method comprising administering a first and a second site-specific endonuclease, or one or more vectors encoding the same in a subject in need thereof wherein the first and second site-specific endonuclease can generate a first and a second double stranded break in a chromosome, thereby generating a first, an intervening second and a third chromosomal fragment, wherein
i) the first fragment comprises a beta globin locus control region (LCR), or a functional fragment thereof; and
ii) the third fragment comprises a sequence encoding HBG1 and/or HBG2;
wherein the length of the second intervening fragment is at least 6 kb, and wherein joining the first fragment to the third fragment results in operably linking the beta globin LCR to the sequence encoding HBG1 and/or HBG2, thereby increasing HBG1 and/or HBG2 protein expression.

19. The method according to claim 18, wherein the haemoglobinopathy is sickle cell disease or beta-thalassemia.


SEQUENCE LISTING (8 sequences)

SEQ ID NO: 1 - DNA, 23 bp, artificial sequence
  guide sequence
cttgtcaaggctattggtcaagg

SEQ ID NO: 2 - DNA, 23 bp, artificial sequence
  guide sequence
attgagatagtgtggggaagggg

SEQ ID NO: 3 - DNA, 20 bp, artificial sequence
  guide sequence
tagtctcatttagtaagcat

SEQ ID NO: 4 - DNA, 20 bp, artificial sequence
  guide sequence
gtggcctctatacctagaag

SEQ ID NO: 5 - DNA, 20 bp, artificial sequence
  guide sequence
ctttatgatgccgtttgagg

SEQ ID NO: 6 - DNA, 20 bp, artificial sequence
  guide sequence
gaggtggagttttagtcagg

SEQ ID NO: 7 - DNA, 20 bp, artificial sequence
  guide sequence
caggatgcttacagggcaga

SEQ ID NO: 8 - DNA, 20 bp, artificial sequence
  guide sequence
gccctgtaagcatcctgctg